(19) United States
(12) Patent Application Publication
(10) Pub. No.: US 2007/0258953 A1
(43) Pub. Date: Nov. 8, 2007

(54) Lactic acid utilising bacteria and their therapeutic use

(76) Inventors: Sylvia Helen Duncan, Aberdeen (GB); Harry James Flint, Aberdeenshire (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 10/550,662

(22) PCT Filed: Mar. 29, 2004
(86) PCT No.: PCT/GB04/01398

§ 371 Date: Nov. 9, 2005

(30) Foreign Application Priority Data
Mar. 27, 2003 (GB) 0307026.5

(51) Int. Cl. A61K 35/74; C12N 1/20

(52) U.S. Cl. 424/93.4; 435/252.1

(57) ABSTRACT

There is provided a method of isolating novel lactic acid utilizing bacteria from human faeces, as well as novel strains so obtained. The use of the novel lactic acid utilising bacteria in therapy, including prophylactic therapy, is described and is of particular relevance for lactic-acidosis, short bowel syndrome and inflammatory bowel disorders such as Crohn's disease and ulcerative colitis. A probiotic comprising the live lactic acid utilising bacteria is also described.

This invention relates to improvements in health and nutrition for both animals and humans following the ingestion of specific bacteria capable of utilising lactic acid.

Under normal conditions the concentration of lactic acid (lactate) in the mammalian gut is very low despite the fact that many bacterial species, such as lactobacilli, streptococci, enterococci and bifidobacteria that reside in the intestine produce this acid in large quantities as a fermentation end product. Lactic acid is also produced by host tissues.

It has been hypothesised that the accumulation of lactic acid is normally prevented by the ability of certain other bacteria that inhabit the gut to consume lactic acid and to use it as a source of energy. The identity of the micro-organisms that are postulated to conduct this metabolic process in the mammalian large intestine has largely not previously been elucidated, Bourriaud et al (2002). Kanauchi et al (1999) revealed that a strain of Bifidobacterium longum was co-incubated with a strain of Eubacterium limosum on germinated barley feedstuff for three days there was a marked increase in acetate formed and a small increase (less than 3 mM) in butyrate formed when compared to the incubations with E. limosum alone.

In the rumen of cattle and sheep the species Selenomonas ruminantium, Veillonella parvula and Megasphaera elsdenii are regarded as the most numerous utilisers of lactate (Gilmour et al., 1994; Wiryawan and Brooker, 1995). The contribution of Megasphaera elsdenii appears to be particularly significant in the rumen, based on the high proportion of carbon flow from lactic acid to propionic acid and this species employs the acrylate pathway for this purpose (Counotte et al., 1981). Megasphaera elsdenii produces a variety of end products including propionate, butyrate, caproate and branched chain fatty acids from lactate—see Ushida et al (2002), Kung and Hession, (1995). This probably reflects the ability of this species to use lactate despite the presence of other carbon sources such as sugars, whereas Selenomonas uses lactic acid only in the absence of other energy sources. This has led to interest in the use of Megasphaera as a probiotic organism that might be added to animal (Kung and Hession, 1995; Ouwerkerk et al., 2002), or even human diets to prevent the harmful accumulation of lactic acid. In ruminant animals (cattle and sheep) accumulation of lactic acid occurs when a large amount of readily fermentable substrate (such as starch and sugars) enters the rumen. Rapid fermentation, particularly by organisms such as Streptococcus bovis, drives down the pH, creating more favourable conditions for the proliferation of lactic acid producing bacteria such as lactobacilli, and S. bovis itself. Normal populations of bacteria capable of utilising lactate (lactate utilisers) are unable to cope with the greatly increased production of lactic acid. Unaided, lactic acid may accumulate to levels that can cause acute toxicity, laminitis and death (Nocek, 1997; Russell and Rychlik, 2001).

Similar events occurring in the large intestine can also cause severe digestive and health problems in other animals, for example in the horse where high lactate levels and colic can result from feeding certain diets.

In humans lactic acid accumulation is associated with surgical removal of portions of the small and large intestine, and with gut disorders such as ulcerative colitis and short bowel syndrome (Day and Abbott, 1999). High concentrations of lactic acid in the bloodstream can cause toxicity (Hove et al., 1994), including neurological symptoms (Chan et al., 1994). Much of this lactic acid is assumed to derive from bacterial fermentation, particularly by bifidobacteria and by lactobacilli and enterococci. Lactic acid can also be produced by host tissues, but the relative contributions of bacterial and host sources are at present unclear.

Conversely, the formation of other acid products, in particular butyric acid (butyrate), is considered to be beneficial as butyric acid provides a preferred energy source for the cells lining the large intestine and has anti-inflammatory effects (Inan et al., 2001, Pryde et al., 2002). Butyrate also helps to protect against colorectal cancer and colitis (Archer et al., 1998; Csordas, 1996).

We have now established a method of isolating novel bacteria that are remarkably active in consuming lactic acid. The bacteria have been isolated from human faeces. Preferably the method allows isolation of bacteria which convert the lactic acid to butyric acid. According to this method several new bacteria that are remarkably active in converting lactic acid to butyric acid have been isolated.

One group of these bacteria is from the newly described genus Anaerostipes caccae (Schwiertz et al., 2002). Although some main characteristics of A. caccae are described in this publication, its ability to use lactate was not reported and has only recently been recognised as described herein.

The invention relates to a method for selecting a strain of lactic acid-utilising bacteria, which method comprises the steps of:
a) providing (for example isolating) a bacterial culture from a human faecal sample;
b) selecting a single colony of bacteria;
c) growing said colony in a suitable medium containing lactic acid; and
d) selecting a strain of bacteria consuming relatively large amounts of lactic acid, all of the above steps being conducted under anaerobic conditions.

In the above method, the reference to “relatively large amounts of lactic acid” is defined as meaning the bacteria used at least 10 mM of D, L or DL lactic acid during growth into stationary phase, per 24 hours at 37° C. in YCFALG or YCFAL medium.

Preferably the strain of lactic acid utilising bacteria also produces high level of butyric acid and the method of the invention may therefore comprise an additional step of:
e) selecting a strain of bacteria producing relatively large quantities of butyric acid.

In the above step the reference to “relatively large quantities of butyric acid” is defined as meaning the bacteria produces at least 10 mM of butyric acid during growth into stationary phase, per 24 hours at 37° C. in YCFALG or YCFAL medium.

Preferably the strain of lactic acid utilising bacterium must be capable of converting lactate produced by another gut bacterium from dietary components such as resistant starch.

Preferably the lactic acid used in step c) is both D- and L-isomers of lactic acid.

Preferably the suitable medium to grow bacteria is nutritionally rich medium in anaerobic Hungate tubes.

Preferably the selected strain of bacteria is re-purified using nutritionally rich medium in anaerobic roll tubes.

A further aspect of the invention is a bacterial strain that produces butyric acid as its sole or predominant fermentation product from lactate and which has been isolated according to the method of the invention described above. Such novel bacterial strains include:

the bacteria Anaerostipes caccae strain L1-92 deposited at NCIMB (National Collections of Industrial, Marine and Food Bacteria in Aberdeen, United Kingdom) under No 13801^{T} on 4 Nov. 2002 and at DSM under No 14662 on 4 Nov. 2002.

the Clostridium indolis bacterial strain Ss2/1 deposited at NCIMB under No 41156 on 13 Feb. 2003;

the bacteria strain SM 6/1 of Eubacterium hallii deposited at NCIMB under No. 41155 on 13 Feb. 2003.

Another aspect of the invention is a strain of bacteria having a 16S rRNA gene sequence which has at least 95% homology to one of the sequences shown in FIG. 1, preferably 97% homology (ie. differs at less than 3% of residues out of approximately 1400 from one of the sequences shown in FIG. 1).

Another aspect of the invention is the use of at least one of the above-mentioned bacterial strains in a medicament or foodstuff.

Another aspect of the invention is a method to promote butyric acid formation in the intestine of a mammal, said method comprising the administration of a therapeutically effective dose of at least one of the above described strains of live butyric acid producing bacteria. The bacterial strain may be administered by means of a foodstuff or suppository or any other suitable method.

Another aspect of the invention is a method for treating diseases associated with a high dosage of lactic acid such as lactic-acidosis, short bowel syndrome and inflammatory bowel disease, including ulcerative colitis and Crohn's disease, which method comprises the administration of a therapeutically effective dose of Anaerostipes caccae or at least one above-mentioned strains of live lactic acid utilising bacteria. Advantageously the strain selected may also produce a high level of butyric acid.

Further, another aspect of the invention is a prophylactic method to reduce the incidence or severity of colorectal cancer or colitis in mammals caused in part by high lactic acid and low butyric acid concentrations, which method comprises the administration of a therapeutically effective dose of at least one above identified strains of live lactic acid utilising bacteria and/or butyric acid producing bacteria mentioned above or of Anaerostipes caccae.

Another aspect of the invention is the use of live Anaerostipes caccae or at least one of the above mentioned lactic acid utilising bacteria as a medicament. Advantageously the strain chosen may produce butyric acid as its sole or predominant fermentation product from lactate. Preferably the bacteria are used in the treatment of diseases associated with high levels of lactic acid such as lactic acidosis, short bowel syndrome and inflammatory bowel disease including ulcerative colitis and Crohn's disease.

According to another aspect of the invention at least one lactate-utilising strain of bacteria as mentioned above or Anaerostipes caccae are used in combination with lactic acid producing bacteria including those such as Lactobacillus spp. and Bifidobacterium spp. or other additives or growth enhancing supplement currently used as probiotics.

The combination of strains would potentially enhance the health-promoting benefits of the lactic acid bacterium by converting its fermentation products (lactic acid alone or lactic acid plus acetic acid) into butyrate. Indeed it is possible that certain health-promoting properties currently ascribed to lactic acid bacteria might actually be due to stimulation of other species such as lactate-consumers in vivo, particularly where probiotic approaches (see below) are used to boost native populations in the gut. Furthermore the presence of the lactic acid producing bacteria in a combined inoculum could help to protect the lactate consumer against oxygen prior to ingestion.

The growth and activity of the novel bacteria may be promoted by means of providing certain growth requirements, required for optimal growth and enzyme expression to the bacteria, present in the animal or human gastrointestinal tract. These bacterial growth enhancing nutrients are often referred to as prebiotics or synbiotics.

Thus the invention provides methods to promote the growth and enzyme expression of the micro-organism and hence removal of lactate and production of butyrate in vivo, for example, via a prebiotic or symbiotic approach (Collins and Gibson, 1999).

Another aspect of the invention is a method for treating acidosis and colic in animals, particularly in ruminants and horses or other farm animals, by administration of a therapeutically effective dose of Anaerostipes caccae or at least one of the lactate utilising bacteria mentioned above. Advantageously the bacteria can be administrated as feed additives.

For the use, prevention or treatment of conditions described herein, the bacteria or prebiotic(s) or symbiotic(s) are preferentially delivered to the site of action in the gastro-intestinal tract by oral or rectal administration in any appropriate formulae or carrier or excipient or diluent or stabiliser. Such modes of delivery may be of any formulation included but not limited to solid formulations such as tablets or capsules; liquid solutions such as yoghurts or drinks or suspensions. Ideally, the delivery mechanism delivers the bacteria or prebiotic or symbiotic without harm through the acid environment of the stomach and through the rumen to the site of action within the gastro-intestinal tract.

Another aspect of the invention is the use of at least one bacterial strain mentioned above or Anaerostipes caccae in a method to produce butyric acid from lactate and acetate. The method includes the fermentation of the above described microorganism selected for both their lactic acid utilising and butyric acid producing abilities in a medium rich in lactate and acetate. The method can be used in industrial processes for the production of butyrate on a large scale.

### BRIEF DESCRIPTION OF THE FIGURE

FIG. 1: Sequence information of 16S rRNA for five lactic acid utilising strains.

FIG. 2: Co-culture experiment. Concentration of SCFA are shown after 24 hours growth in YCFA medium with 0.2% starch as energy source (values for acetate, initially present in the medium, are shown on a 10 fold reduced scale). Butyrate production by A. caccae L1-92, and by E. hallii L2-7 and SM 6/1, is stimulated by co-culture with B. adoloscentis L2-32, while L-lactate disappears from the co-cultures.

FIG. 3: SCFA formation and lactate utilisation for new and existing isolates. Acids produced or consumed during anaerobic growth are shown for strains incubated for 24 hours: a) YCFA medium containing 35 mM DL lactate (YCFAL); b) YCFA medium containing 10 mM glucose and 35 mM DL lactate (YCFALG); c) YCFA medium with no addition. Carbon recoveries (%) for growth on lactate, and lactate plus glucose, respectively, were as follows: SM 6/1 (94.6, 76.4); SL 6/1/1 (100.2, 78.7); L1-92 (96.2, 97.9); SS2/1 (92.1, 90.1); SSC/2 (104.4, 96.9); SR1/1 (103, 93.8). This suggests that there may be unidentified fermentation products in the case of SM 6/1, SL 6/1/1 and SS3/4 when grown on glucose plus lactate.

FIG. 4: Time course of SCFA formation and growth in batch culture of E. hallii L2-7 on media containing DL lactate, glucose, or DL lactate plus glucose.

FIG. 5: Time course of SCFA formation and growth in batch culture of strain SS2/1 on media containing DL lactate, glucose, or DL lactate plus glucose.

### DETAILED DESCRIPTION

The experimental work performed shows the following:
1. Certain human colonic anaerobic bacteria, including A. caccae strains, are strong and efficient utilisers of lactic acid.
2. Certain human colonic anaerobic bacteria, including A. caccae strains, are strong and efficient producers of butyric acid.
3. Certain human colonic anaerobic bacteria, including A. caccae strains, convert lactic acid to butyric acid.

### EXAMPLE 1

### Isolation and Characterisation of Bacteria

A faecal sample was obtained from a healthy adult female volunteer that had not received antibiotics in the previous 6 months. Whole stools were collected, and 1 g was mixed in 9 ml anaerobic M2 diluent. Decimal serial anaerobic dilutions were prepared and 0.5ml inoculated into roll tubes by the Hungate technique, under 100% CO₂ (Byrant, 1972).

Bacterial strains were isolated by selection as single colonies from the nutritionally rich medium in anaerobic roll tubes as described by Barcenilla et al. (2000). The isolates were grown in M2GSC broth and the fermentation end products determined. Butyrate producing bacteria were re-purified using roll tubes as described above. Strains L1-92, S D8/3, S D7/11, A2-165, A2-181, A2-183, L2-50 and L2-7 were all isolated using this medium. Omitting rumen fluid and/or replacing the sugars with one additional carbon source such as DL lactate increased the selectivity of the roll tube medium and this medium was used to isolate strain S D6 1L/1. Strains G 2M/1 and SM 6/1 were isolated from medium where DL-lactate was replaced with mannitol (0.5%). Separately, non-rumen fluid based media routinely used for isolating Selenomonas sp., namely Ss and Sr medium (Atlas, 1997) was used to isolate other strains. Inoculating Sr medium roll tubes with dilutions of faecal samples resulted in the isolation of strain Sr1/1 while the Ss medium resulted in the isolation of strains Ss2/1, Ss3/4 and Ssc/2.

### EXAMPLE 2

### A. caccae and other Human Colonic Bacterial Isolates Consumes Lactic Acid and Acetic Acid and Produces Butyric Acid when Grown in Rumen Fluid

Table 1 summarises the fermentation products formed by twelve strains of anaerobic bacteria when grown under 100% CO₂ in a rumen fluid-containing medium containing 0.5% lactate (M2L) or 0.5% lactate, 0.2% starch, 0.2% cellobiose and 0.2% glucose (M2GSCL) as the energy sources. Ten of these strains were isolated from human faeces as described above in Example 1. Strains 2221 and NCIMB8052 are stock collection isolates not from the human gut and are included for comparison. Table 1 demonstrates that three strains, L1-92 (A. caccae), SD6 1L/1 and SD 6M/1 (both E. hallii-related) all consumed large amounts of lactate (>20 mM) on both media examined, M2L and M2GSCL, and produced large quantities of butyric acid. A. caccae L1-92 in particular consumed large amounts of lactate and produced large amounts of butyrate. Acetate is also consumed by all three strains. The other 9 butyrate producing bacteria tested either consumed relatively small amounts of lactate, or consumed no lactate, on this medium. L-lactate concentrations were determined enzymatically and glucose concentrations were determined by the glucose oxidase method (Trinder, 1969). Analyses were conducted in a robotic clinical analyser (Kone analyser, Konelab Corporation, Finland).

TABLE 1
Table 1. Comparison of human faecal isolates for the ability to utilise
(negative values) or produce (positive values) lactate on a rumen fluid
based medium (M2) supplemented with lactate (M2L) and lactate plus glucose,
cellobiose and soluble starch (0.2% each) (M2GSC).

| Strain ID | Closest relative | Medium | Formate | Acetate | Butyrate | Lactate |
|---|---|---|---|---|---|---|
| S D8/3 | Adhufec 406*+ | M2L | 1.15 | 0.97 | | −3.94 |
| S D8/3 | | M2GSCL | 21.66 | 0.77 | 10.88 | 6.43 |
| SL 6/1/1 | E. hallii | M2L | | −19.74 | 35.48 | −32.41 |
| SL 6/1/1 | | M2GSCL | | −9.78 | 22.58 | −21.85 |
| SM 6/1 | E. hallii | M2L | 0.79 | −19.01 | 31.73 | −23.72 |
| SM 6/1 | | M2GSCL | 1.31 | −5.06 | 22.77 | −28.42 |
| G 2M/1 | HucA19* | M2L | | 2.82 | 7.97 | 23.66 |
| G 2M/1 | | M2GSCL | | 0.01 | 12.94 | 9.52 |
| S D7 11/1 | ND^{#} | M2L | | 0.51 | 0.08 | 7.58 |
| S D7 11/1 | | M2GSCL | 1.85 | 0.43 | 4.84 | −10.25 |
| 2221 | But. Fibrisolvens | M2L | 1.37 | −3.61 | 1.57 | 21.57 |
| 2221 | | M2GSCL | 19.4 | −5.57 | 18.02 | 11.75 |
| 8052 | Cl. acetobutylicum | M2L | | −12.42 | 19.31 | −0.87 |
| 8052 | | M2GSCL | 0.13 | −1.79 | 18.00 | −5.80 |
| A2-165 | F. prausnitzii | M2L | 1.98 | 0.62 | 3.56 | 2.94 |
| A2-165 | | M2GSCL | 17.47 | −6.97 | 18.38 | −5.65 |
| A2-183 | Roseburia sp. | M2L | | 0.86 | 1.84 | 10.63 |
| A2-183 | | M2GSCL | −0.15 | −12.70 | 18.23 | 5.33 |
| A2-181 | Roseburia sp. | M2L | 0.58 | −0.26 | 1.75 | |
| A2-181 | | M2GSCL | 0.33 | −11.05 | 18.68 | 5.22 |
| L2-50 | Coprococcus sp. | M2L | 1.06 | 2.32 | 0.52 | 0.43 |
| L2-50 | | M2GSCL | 19.37 | 4.47 | 7.60 | 3.41 |
| L1-92 | Anaerostipes caccae | M2L | | −29.42 | 37.00 | −25.60 |
| L1-92 | | M2GSCL | 0.63 | −27.03 | 44.78 | −45.48 |

*clone library sequence, uncultured (Hold et al., 2002)
+clone library sequence, uncultured (Suau et al., 1999)
#ND not determined

### EXAMPLE 3

### A. caccae and other Human Colonic Bacterial Isolates Consumes Lactic Acid and Acetic Acid and Produces Butyric Acid when Grown in Rumen Fluid Free Medium

Table 2a shows the utilisation and production of formate, acetate, butyrate, succinate and lactate, on this occasion performed using the rumen fluid-free medium YCFA (Duncan et al. 2002) containing no added energy source, or with 32 mM lactate (YCFAL) or lactate plus 23 mM glucose (YCFALG) as added energy sources. Separately Table 2b reveals the levels of the two isomers of lactate (D and L) remaining at the end of the incubations and the concentration of glucose metabolised during the incubations. Five additional new lactate-utilising isolates were discovered using the semi-selective medium as described earlier and are included in Tables 2a and 2b, although one of these (Ss 3/4) proved to-consume a relatively small amount of lactate only on the YCFAL medium (Table 2a). Analysis of the consumption of the D and L isomers reveals that three strains (Ss2/1, Ssc/2 and Sr1/1) preferentially consumed D lactate. Partial repression of lactate consumption by glucose was observed on this medium with A. caccae L1-92, and almost complete repression for SL 6/1/1 and Ss 3/4. The previously isolated E. hallii strain L2-7 (Barcenilla et al., 2000) behaved in a similar manner to SL 6/1/1. The higher glucose concentration in this medium compared with M2GSCL is likely to explain the difference in behaviour of A. caccae compared with Table 1. The remaining five strains showed no evidence of repression of lactate utilisation in the presence of glucose although it is possible they use the glucose before switching to lactate. Butyrate levels exceeding 30 mM were obtained for four strains on YCFALG medium.

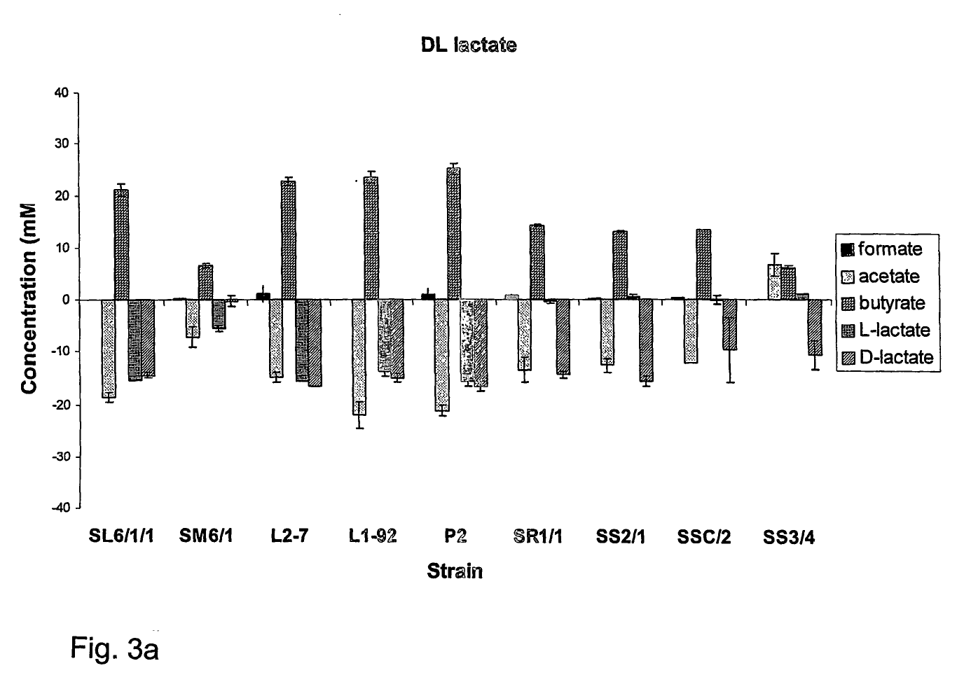

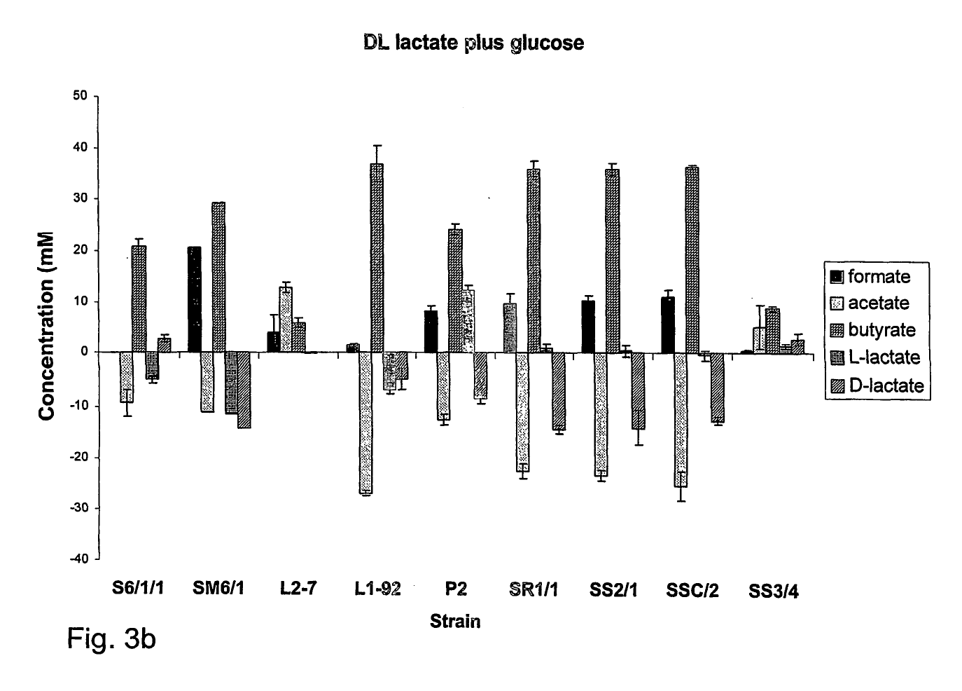

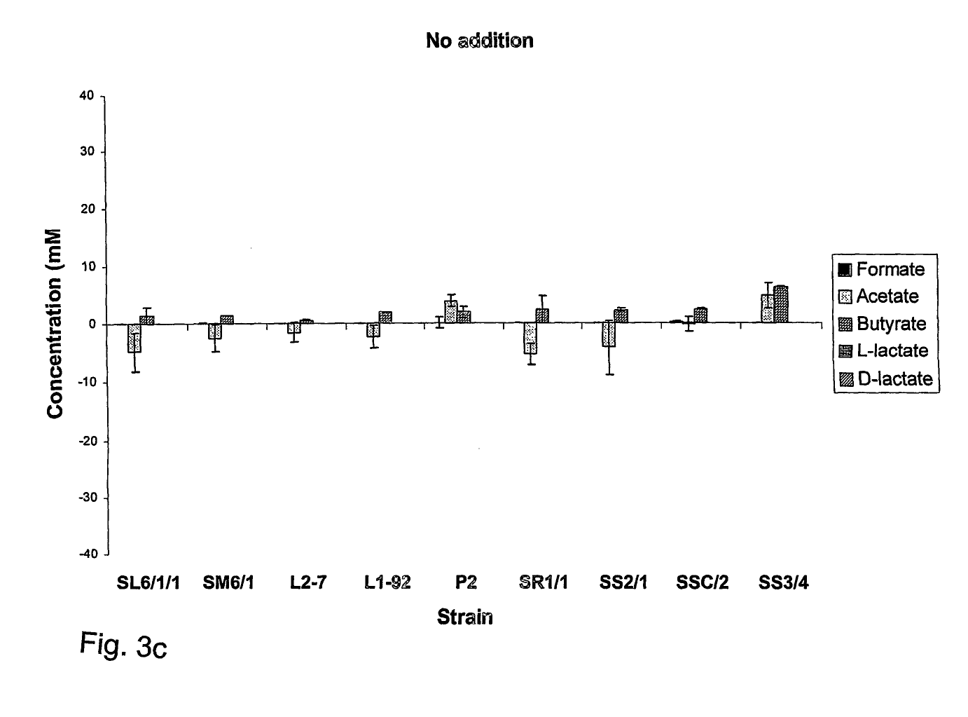

Results : The three E. hallii-related strains (L-27, SL 6/1/1, SM 6/1) and the two A. caccae strains (L1-92 and P2) were able to use both the D and L isomers of lactate during growth either on DL lactate or DL lactate plus glucose (FIG. 3). The four remaining new isolates SR1/1, SSC/2, SS2/1 and SS3/4 however showed a strong preference for using D-lactate. In most strains, except SS3/4 and L2-7, some utilisation of lactate was detectable following 24 hours incubation even when glucose was initially present in the medium (FIG. 3).

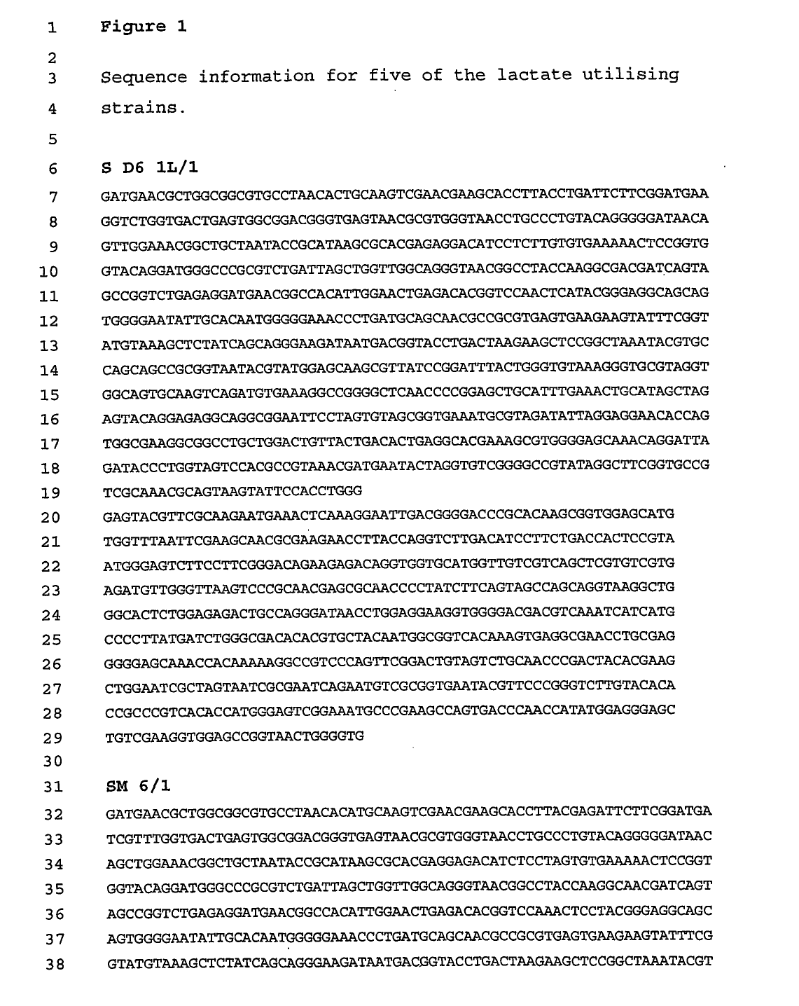

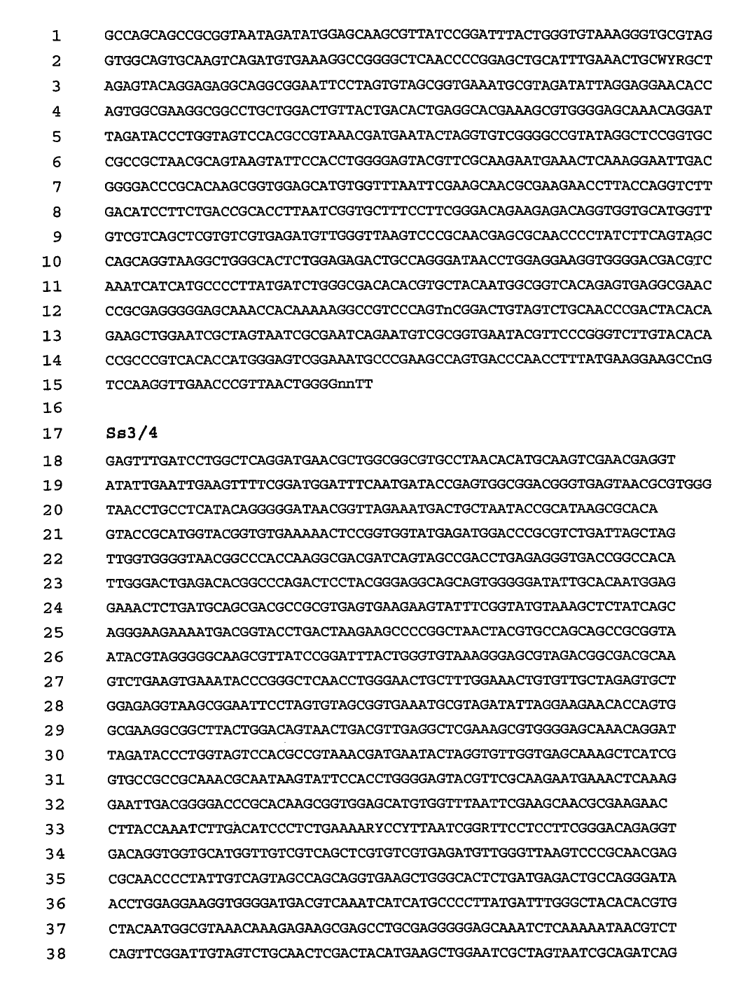

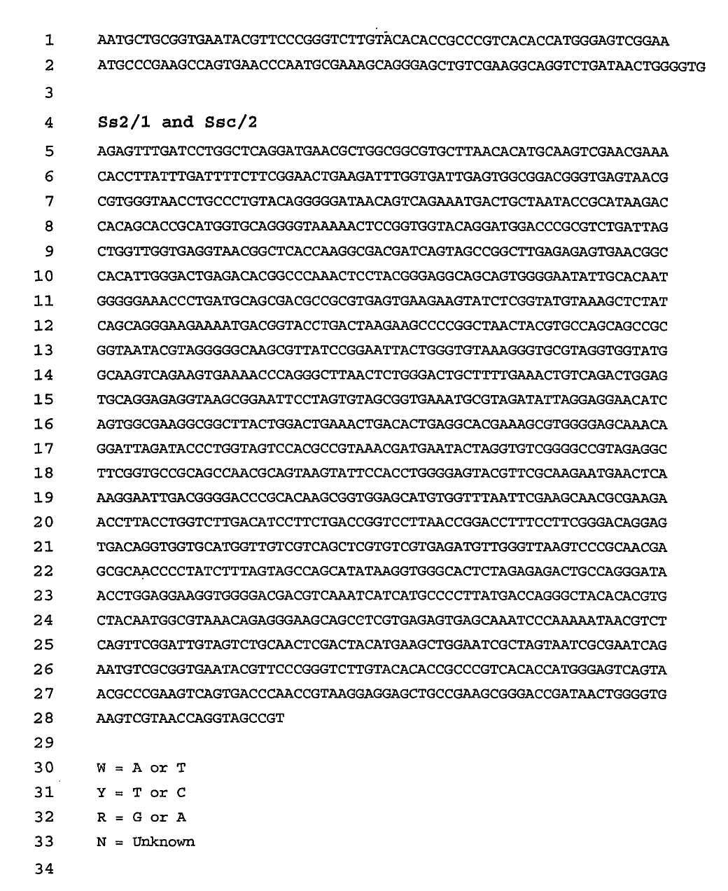

TABLE 2a
Table 2a. Fermentation products formed or utilised (U as indicated by minus values) by human gut isolates incubated on yeast extract-casitone-fatty
acids medium (YCFA); YCFA supplemented with lactate (YCFAL); and YCFA supplemented with glucose and lactate (YCFALG). The initial
concentration of glucose added to the medium was 23 mM and 32 mM lactate was added that contained 15.5 mM L-lactate. ^{a}Strain identity
is based on 16S rRNA sequence information (% identical residues with closest relative is shown). See Figure 1 for sequence information.
All strains except 2221 and 8052 (Table 1) were isolated as described in Example 1.

| Strain ID | Closest relative^{a} | Isolation Medium | Medium | Formate | Acetate P/U | Butyrate | Succin | Lactate P/U |
|---|---|---|---|---|---|---|---|---|
| Ss2/1 | Cl. indolis (95%) | Selenomonas selective | YCFA | 0.02 ± 0.04 | −4.25 ± 4.68 | 2.24 ± 0.26 | | 0.39 ± 0.03 |
| | | | YCFAL | 0.18 ± 0.02 | −12.51 ± 1.27 | 12.98 ± 0.19 | | −15.27 ± 2.53 |
| | | | YCFALG | 10.10 ± 1.05 | −24.32 ± 1.03 | 35.69 ± 1.13 | | −13.95 ± 2.70 |
| Sr 1/1 | Ruminococcus obeum | Selenomonas | YCFA | | −5.42 ± 1.77 | 2.33 ± 0.03 | | 0.36 ± 0.12 |
| | HucB 12* | ruminantium | YCFAL | 0.76 ± 0.19 | −13.35 ± 2.27 | 14.15 ± 0.17 | | −15.04 ± 0.89 |
| | | | YCFALG | 9.53 ± 2.03 | −22.47 ± 1.40 | 35.77 ± 1.50 | | −13.71 ± 0.40 |
| SL 6/1/1 | E. hallii | M2 + 0.5% lactate | YCFA | | −4.96 ± 3.26 | 1.42 ± 0.23 | | |
| | HucA 15* | | YCFAL | | −18.51 ± 0.96 | 21.06 ± 1.06 | | −29.93 ± 0.60 |
| | | | YCFALG | | −9.22 ± 2.52 | 20.78 ± 1.52 | | −2.43 ± 0.70 |
| SM 6/1 | E. hallii (98%) | M2 + 0.5% mannitol | YCFA | 0.09 ± 0.03 | −2.61 ± 2.36 | 1.42 ± 0.05 | | |
| | | | YCFAL | 0.21 ± 0.1 | −7.20 ± 2.08 | 6.54 ± 0.43 | | −6.27 ± 1.27 |
| | | | YCFALG | 20.68± | −10.95± | 29.2± | | −25.82± |
| Ss 3/4 | Ruminococcus gnavus | Selenomonas selective | YCFA | | 4.75 ± 2.20 | 6.10 ± 0.27 | | 1.09 ± 0.47 |
| | HucA19* | | | | | | | |
| Ss 3/4 | Ruminococcus gnavus | Selenomonas selective | YCFA | | 4.75 ± 2.20 | 6.10 ± 0.27 | | 1.09 ± 0.47 |
| | HucA19* | | YCFAL | | 6.68 ± 2.09 | 6.19 ± 0.34 | | −9.78 ± 2.56 |
| | | | YCFALG | 0.54 ± 0.13 | 5.06 ± 4.28 | 8.66 ± 0.53 | | 3.86 ± 1.09 |
| Ssc/2 | Cl. indolis (95%) | Selenomonas selective | YCFA | 0.25 ± 0.04 | −0.16 ± 1.32 | 2.37 ± 0.09 | | 0.48 ± 0.03 |
| | | | YCFAL | 0.36 | −12.12 | 13.49 | | −13.78 |
| | | | YCFALG | 10.98 ± 1.27 | −25.35 ± 2.87 | 36.10 ± 0.49 | | −13.34 ± 1.28 |
| L1-92 | A. caccae | M2GSC | YCFA | 0.00 ± 0.08 | −2.35 ± 2.03 | 1.99 ± 0.09 | | |
| | (type strain) | | YCFAL | −0.05 ± 0.10 | −21.98 ± 2.45 | 23.35 ± 1.16 | | −28.92 ± 0.54 |
| | | | YCFALG | 1.49 ± 0.13 | −26.83 ± 0.58 | 36.81 ± 3.61 | | −12.01 ± 1.32 |
| L2-7 | E. hallii | M2GSC | YCFA | 0.02 ± 0.01 | −1.58 ± 1.73 | 0.63 ± 0.03 | | 0.00 ± 0.00 |
| | | | YCFAL | 1.09 ± 1.55 | −14.77 ± 0.93 | 22.58 ± 0.76 | | −30.47 ± 0.00 |
| | | | YCFALG | 3.93 ± 3.38 | 12.78 ± 0.94 | 5.80 ± 0.97 | | 1.67 ± 0.47 |

*clone library sequences, uncultured (Hold et al., 2002)

TABLE 2b
Total lactate (mM) remaining in the tubes at the end of the 24 h incubation
period and separately the concentration of the two forms D and L. Total
glucose (gluc) metabolised during growth also recorded (mM).

| Strain | | | | | | |
|---|---|---|---|---|---|---|
| number | Closest relative | Medium | Total lact. | L-lact | D-lact | Gluc used |
| Ss2/1 | Cl. indolis (95%) | YCFA | 0.84 ± 0.02 | | | |
| | | YCFAL | 17.08 ± 2.53 | 16.07 ± 0.40 | 1.01 ± 2.15 | |
| | | YCFALG | 18.40 ± 2.70 | 15.90 ± 1.06 | 2.50 ± 3.30 | 22.1 ± 0.0 |
| Sr 1/1 | Huc B12* | YCFA | 0.81 ± 0.12 | | | |
| | | YCFAL | 17.31 ± 0.89 | 15.05 ± 0.34 | 2.26 ± 0.68 | |
| | | YCFALG | 18.64 ± 0.40 | 16.37 ± 0.79 | 2.27 ± 0.71 | 22.0 ± 0.2 |
| SL 6/1/1 | E. hallii | YCFA | 0.00 ± 0.00 | | | |
| | Huc A15* | YCFAL | 2.42 ± 0.60 | 0.21 ± 0.10 | 2.21 ± 0.51 | |
| | | YCFALG | 29.92 ± 0.07 | 10.65 ± 0.69 | 19.27 ± 0.79 | 22.1 ± 0.1 |
| SM 6/1 | E. hallii (98%) | YCFA | 0.00 ± 0.00 | | | |
| | | YCFAL | 26.08 ± 1.27 | 9.94 ± 0.50 | 16.14 ± 1.06 | |
| | | YCFALG | 6.57 ± 0.16 | 4.02 ± 2.26 | 2.55 ± 2.32 | 22.1 ± 0.1 |
| Ss 3/4 | HucA19* (new species | YCFA | 1.54 ± 0.47 | | | |
| | to be named) | YCFAL | 22.58 ± 2.55 | 16.56 ± 0.12 | 6.02 ± 2.65 | |
| | | YCFALG | 6.57 ± 0.16 | 4.02 ± 2.26 | 2.55 ± 2.32 | 22.1 ± 0.1 |
| Ss 3/4 | HucA19* (new species | YCFA | 1.54 ± 0.47 | | | |
| | to be named) | YCFAL | 22.58 ± 2.55 | 16.56 ± 0.12 | 6.02 ± 2.65 | |
| | | YCFALG | 36.21 ± 1.09 | 16.95 ± 0.87 | 19.26 ± 1.91 | 16.6 ± 0.6 |
| Ssc/2 | A. caccae (L1-92) | YCFA | 0.96 ± 0.08 | | | |
| | | YCFAL | 22.39 ± 6.63 | 15.40 ± 0.78 | 6.99 ± 6.10 | |
| | | YCFALG | 19.01 ± 1.28 | 15.08 ± 0.93 | 3.93 ± 0.68 | 22.2 ± 0.0 |
| L1-92 | A. caccae (type strain) | YCFA | 0.0 ± 0.0 | | | |
| | | YCFAL | 3.43 ± 0.54 | 1.84 ± 0.85 | 1.59 ± 0.87 | |
| | | YCFALG | 20.34 ± 1.32 | 8.63 ± 0.72 | 11.71 ± 2.01 | |
| L2-7 | E. hallii | YCFA | 0.00 ± 0.00 | | | |
| | | YCFAL | 0.00 ± 0.00 | | | |
| | | YCFALG | 31.93 ± 0.47 | 15.43 ± 0.12 | 16.50 ± 0.30 | 11.99 ± 0.71 |

*clone library sequence, uncultured (Hold et al., 2002)

### EXAMPLE 4

### 16S rRNA Sequencing of New Isolates and Phylogenetic Relationships

Cell pellets from 1 ml cultures grown on M2GSC medium (24 h) that were resuspended in 50 μl of sterile d.H₂O served as templates for PCR reactions (0.5 μl per 50 μl of PCR reaction). 16S rRNA sequences were amplified with a universal primer set, corresponding to positions 8-27 (27f, forward primer, AGAGTTTGATCMTGGCTCAG) and 1491-1511 (rP2, reverse primer ACGGCTACCTTGTTACGACTT) of the Escherichia coli numbering system (Brosius, 1978; Weisberg, 1991) with a MgCl₂ concentration of 1.5 mM. PCR amplifications were performed using the following conditions: initial denaturation (5 min at 94° C.), then 30 cycles of denaturation (30 s at 94° C.), annealing (30 s at 51° C.), and elongation (2 min at 72° C.), and a final extension (10 min at 72° C.). The amplified PCR products were purified using QIA quick columns (Qiagen GmbH, Germany) according to manufacturer's instructions and directly sequenced using a capillary sequencer (Beckman) with primers 27f, rP2, 519f(CAGCMGCCGCGGTAATWC) and 519r (GWATTACCGCGGCKGCTG) (corresponding to positions 518-535 of the E. coli numbering system) and 926f (AAACTCAAAKGAATTGACGG) and 926r (CCGTCAATTCMTTTRAGTTT) corresponding to positions 906-925). Two independent PCR products were sequenced per strain.

Similarity of the 16S rRNA sequences (minimum 1444 bases) of the isolates with other organisms was compared with all sequence data in GenBank using the BLAST algorithm (Altschul, 1990).

### EXAMPLE 5

### Co-Culture of Lactate Utilisers With Bifiidobacterium adolescentis

Three lactate utilising strains, Anaerostipes caccae L1-92 and two strains of Eubacterium hallii (SM 6/1 and L2-7) were incubated alone and in co-culture with B. adolescentis L2-32 on YCFA medium modified to contain reduced casitone (0.1%) and 0.2% soluble starch as an added energy source. The inoculated tubes were incubated for 24 h at 37° C. B. adolescentis L2-32 was enumerated on Mann Ragosa Sharpe (MRS) medium containing 2.0% agar with a final concentration of 0.5% propionate and the three butyrate producing strains, were enumerated on M2 medium containing 0.5% DL lactate.

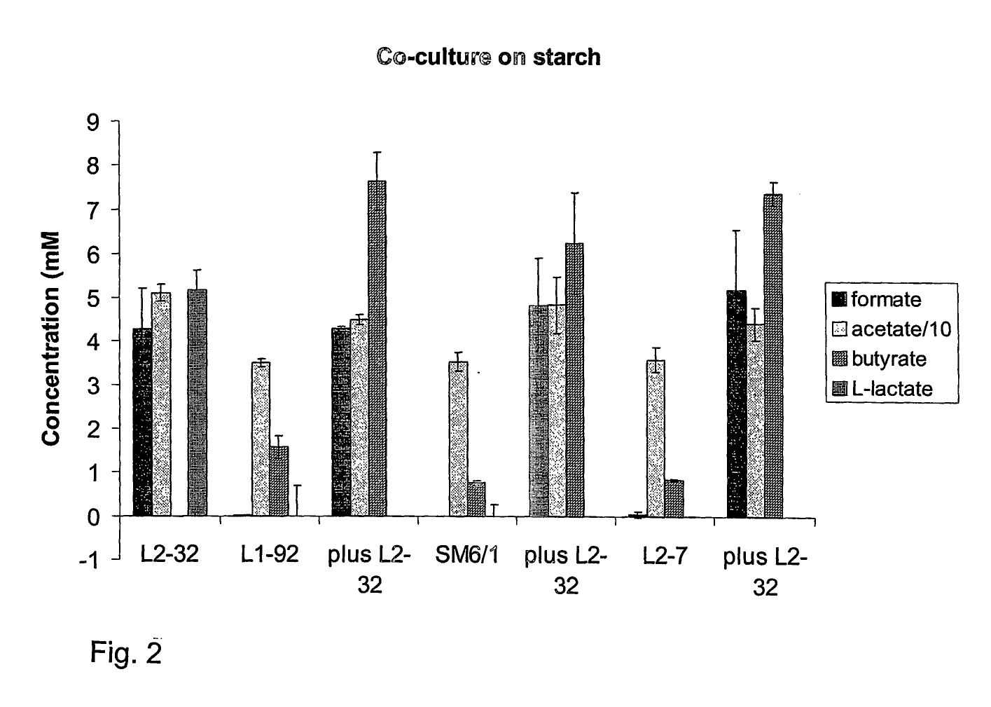

Results: In most human diets, resistant starch is considered to be the most important energy source for microbial growth in the large intestine (Topping, 2001). The major amylolytic species in the human colon are generally considered to be Bacteroides and Bifidobacterium spp. (MacFarlane, 1986; Salyers, 1977). Bifidobacteria produce acetate and lactate from carbohydrate substrates, typically in the molar ratio of 3:2. Since the lactate utilisers isolated here either do not utilise starch or utilised it weakly, as a growth substrate in pure culture, it was of interest to co-culture them with a starch-degrading Bifidobacterium strain in order to establish whether they could remove the lactate formed. The recently isolated, actively amylolytic B. adolescentis strain L2-32 was used for these experiments. As shown in Tables 3a, 3b and FIG. 2, co-culture with any one of three lactate utilisers tested, with starch as the growth substrate, resulted in complete conversion of the L-lactate, and some of the acetate, formed by B. adolescentis L2-32 into butyric acid. This corresponded with greatly increased growth of the lactate utilisers in the presence of the B. adoliscentis L2-32, as determined by selective plating. Viable counts (cfu ml⁻¹) after 24 hours growth for L1-92, SM 6/1 and L2-7 were, respectively, 2.4×10⁸, 1.0×10⁷ and 8.0×10⁶, in the absence of B. adolescentis, and 1.7×10⁹, 6.8×10⁸ and 5.4×10⁹, in the presence of B. adolescentis L2-32. Growth of B. adolescentis L2-32 was unaffected by co-culture (mean 4.3×10⁸ cfu ml⁻¹). There may have been some contribution of starch hydrolysis products that escape uptake by the B. adolescentis L2-32, in addition to lactate and acetate, to the growth of the lactate utilisers. This might account for the apparent effectiveness of E. hallii SM 6/1 in co-culture, even though this strain used rather little in pure culture when supplied with lactate alone.

TABLE 3a
Fermentation profiles for Bifidobacterium adolescentis L2-32 and three lactate utilisers
when incubated alone or in co-culture for 24 hours at 37° C. on modified YCFA
medium (modified to contain 0.1% casitone) containing 0.2% soluble starch.

| Culture/ | | | | Total | |
|---|---|---|---|---|---|
| co-culture | Formate | Acetate | Butyrate | Lactate | L-Lactate |
| L2-32 | 4.29 ± 0.92 | 51.04 ± 5.44 | 0 | 5.00 ± 0.09 | 5.16 ± 0.45 |
| L1-92 | 0.01 ± 0.01 | 34.99 ± 0.93 | 1.57 ± 0.26 | 0.40 ± 0.69 | 0 |
| SM 6/1 | 0 | 35.25 ± 2.15 | 0.75 ± 0.06 | 0.27 ± 0.27 | 0 |
| L2-7 | 0.04 ± 0.06 | 35.70 ± 0.44 | 0.83 ± 0.02 | 0 | 0 |
| L2-32 + L1-92 | 4.29 ± 0.04 | 44.82 ± 1.13 | 7.62 ± 0.66 | 0.61 ± 0.53 | 0 |
| L2-32 + SM 6/1 | 4.81 ± 1.08 | 48.17 ± 6.47 | 6.23 ± 1.15 | 0 | 0 |
| L2-32 + L2-7 | 5.16 ± 1.37 | 43.88 ± 3.74 | 7.35 ± 0.27 | 0.36 ± 0.01 | 0 |

TABLE 3b
Total viable counts (cfu per ml) of Bifidobacterium adolescentis L2-32
and three lactate utilisers following 24 hours at 37° C. in monoculture
and co-culture. Bifidobacterium adolescentis L2-32 was selected for on
MRS + 0.25% propionate roll tubes and the butyrate producing/lactate
utilisers were selected for on M2 + 0.5% lactate roll tubes following
incubation for 24 hours at 37° C.

| Culture/ | B. adolescentis | Butyrate producer/ |
|---|---|---|
| Co-culture | L2-32 | lactate utiliser |
| L2-32 | 3.8 × 10⁸ | |
| L1-92 | | 2.4 × 10⁸ |
| S M6/1 | | 1.0 × 10⁷ |
| L2-7 | | 8.0 × 10⁶ |
| L2-32 + L1-92 | 6.4 × 10⁸ | 1.7 × 10⁹ |
| L2-32 + SM 6/1 | 3.8 × 10⁸ | 6.8 × 10⁸ |
| L2-32 + L2-7 | 3.2 × 10⁸ | 5.4 × 10⁹ |

### EXAMPLE 6

### Time Course of Lactate Utilisation

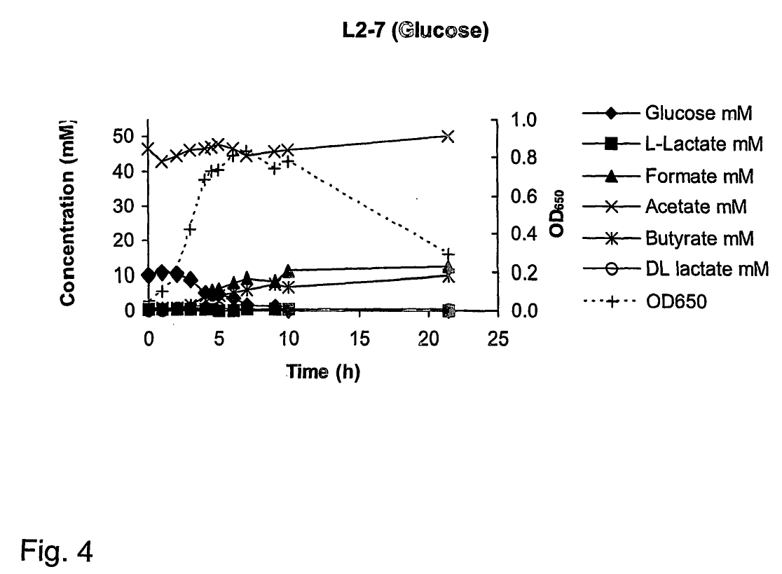

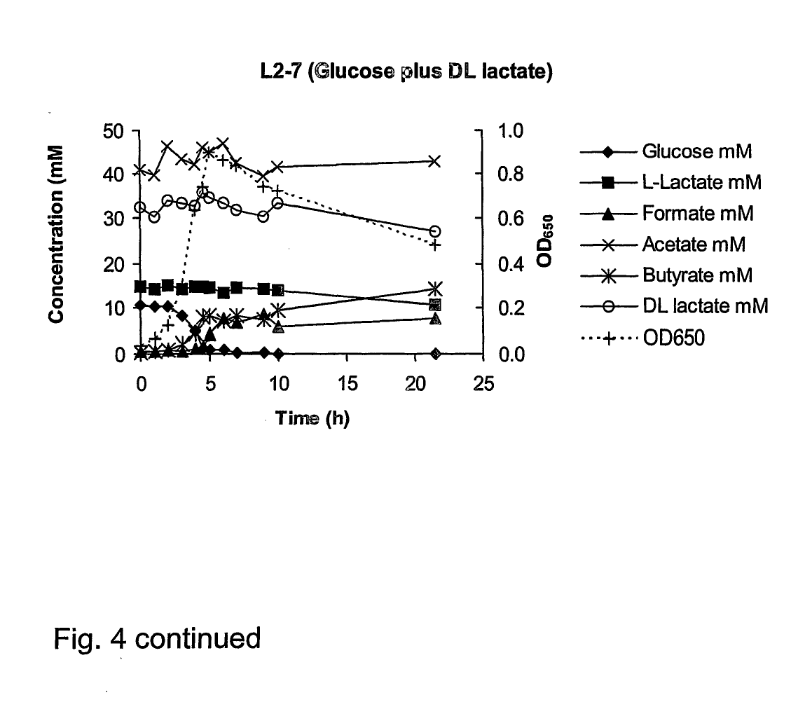

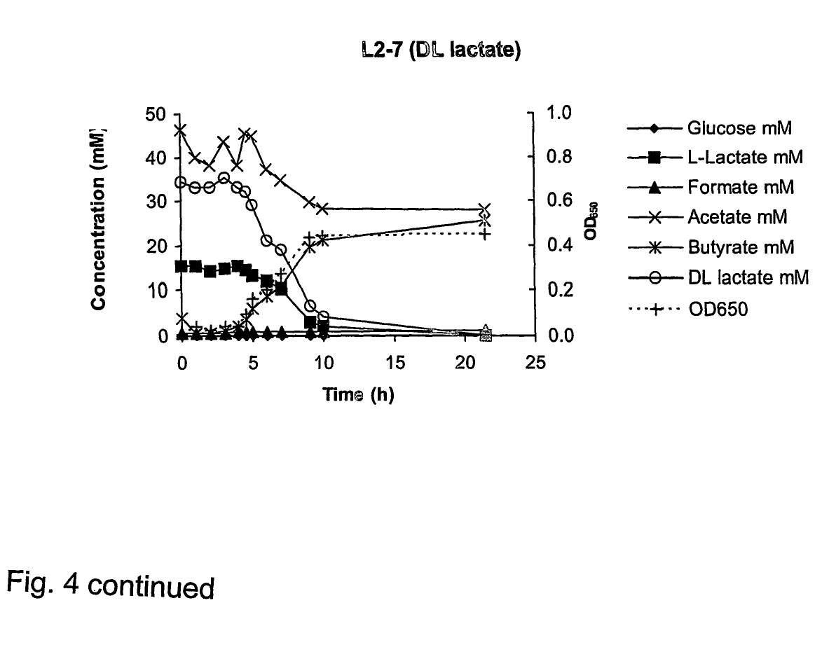

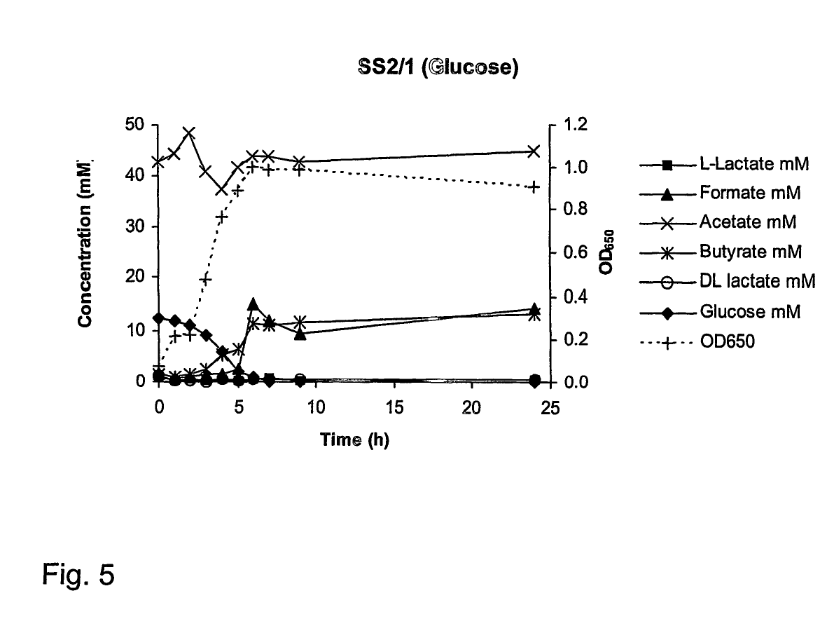

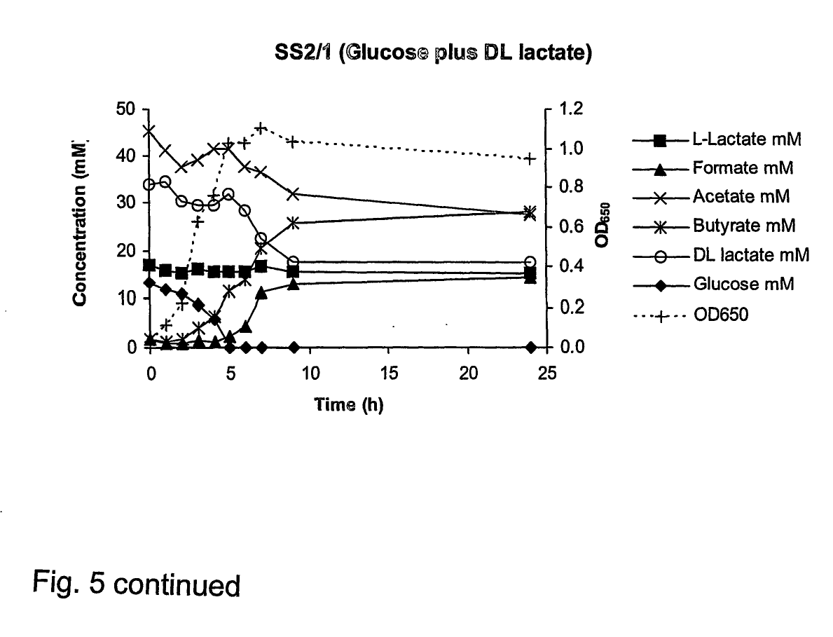

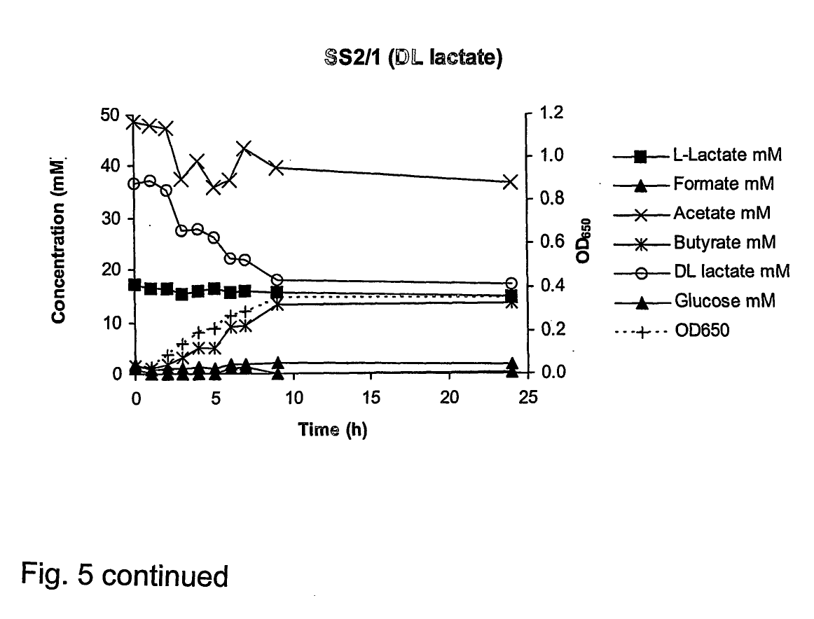

Time courses were followed in batch culture for growth on glucose, lactate or glucose and lactate (FIGS. 4, 5). E. hallii L2-7 when grown with DL-lactate used all of the added lactate together with some acetate, producing more than 20 mM butyrate (FIG. 4). Less butyrate, but significant formate, was produced during growth on glucose, or on glucose plus lactate, and lactate utilisation was almost abolished by the presence of glucose. Hydrogen production in 24 hours was 12 μm ml⁻¹ for growth on glucose, 15.5 μmol ml⁻¹ for growth on lactate and 10.9 μmol ml⁻¹ for growth on glucose plus lactate. A. caccae L1-92 similarly produce larger quantities of butyrate when grown on lactate compared with growth on glucose, when formate was also a product. This strain was able to use lactate once glucose had been exhausted, following inoculation into glucose plus lactate medium.

Strain SS2/1 is likely to represent a new species, since its closest relative (95% identity in 16S rRNA sequence) is the non-butyrate producing Clostridium indolis. This strain was able to use D-, but not L-, lactate following glucose exhaustion in lactate plus glucose medium (FIG. 5). Again formate was not a significant product when lactate was the sole energy source but 4.7 μmol ml⁻¹ hydrogen was formed.

### SUMMARY

A. caccae strain L1-92 was able to consume up to 30 mM DL lactate, along with 20-30 mM acetate during batch culture incubation for 24 hours at 37° C. with the production of >20 mM, and up to 45 mM butyrate; this occurred also when glucose was added as an alternative energy source (Table 1). Lactate or lactate plus glucose thus resulted in very much higher production of butyrate than observed with 23 mM glucose alone, when only <15 mM butyrate was formed. Furthermore none of the 74 strains screened previously by Barcenilla et al. (2000) produced more than 25 mM butyrate when tested in M2GSC medium. Lactate consumption is not a general characteristic of butyrate-producers, and six of the strains screened in Table 1 failed to consume lactate in M2GSCL medium.

Six further strains that are highly active lactate utilisers (defined for example as net consumption of at least 10 mM of lactate during growth to stationary phase or for 24 hours in YCFALG or YCFAL medium at 37° C.—see Table 2a) were obtained following deliberate screening of new human faecal isolates for lactate utilisation. At least two of these (SL 6/1/1 and SM 6/1—Tables 1, 2) are related to Eubacterium hallii. (Table 2a), based on determination of their 16S rDNA sequences. These isolates again consume large quantities of lactate and produce high levels of butyrate in vitro. With one exception where considerable glucose repression occurred (strain SL 6/1/1), significant lactate utilization occurred in the presence of glucose (Table 2). Three strains (Ss 2/1, Sr 1/1 and Ssc/2) showed preferential utilization of D-lactate, whereas the two E. hallii-related strains SM 6/1, SL 6/1/1 and A. caccae L1-92 utilise both isomers (Table 2b). The two stereoisomers differ in their toxicity in the human body, with the D-isomer being regarded as the more toxic (Chan et al., 1994, Hove et al., 1995). The present invention thus provides a means of utilising both D and L lactate isomers or preferentially utilising D-lactate in preference to L-lactate.

A. caccae and newly isolated bacteria related to E. hallii and Cl. indolis were shown to consume up to 30 mM DL, D or L lactate, along with 20-30 mM acetate during batch culture incubation and convert this energy in to production of at least 20 mM, and up to 45 mM butyrate. Furthermore, these strains were shown to convert all of the L-lactate produced by a starch-degrading strain of Bifidobacterium adolescentis into butyrate when grown in culture. This is the first documentation demonstrating the conversion of lactate to butyrate by human colonic bacteria, some of which are likely to be new species.

### REFERENCES

Altschul, S. F., Gish, W., Miller, W., Myers, E. W. and Lipman, D. J. (1990) Basic local alignment search tool. J. Mol. Biol. 215, 403-410.
Archer, S. Y., Meng, S. F., Sheh, A. and Hodin, R. A. (1998). p21 (WAF1) is required for butyrate mediated growth inhibition of human colon cancer cells. Proc. Natl. Acad. Sci. USA, 95, 6791-6796.
Atlas, R. M. (1997). Handbook of microbiological media (2^{nd} edition). Ed. L. C. Park. CRC Press, Cleveland, Ohio.
Barcenilla, A., Pryde, S. E., Martin, J. C., Duncan, S. H., Stewart, C. S., Henderson, C. and Flint, H. J. (2000). Phylogenetic relationships of dominant butyrate producing bacteria from the human gut. Appl. Environ. Microbiol., 66, 1654-161.
Bourriaud, C., Akoka, S., Goupry, S., Robins, R., Cherbut, C. and Michel, C. (2002). Butyrate production from lactate by human colonic microflora. Reprod. Nutr. Develop., 42, (Suppl. 1). S55.
Brosius, J., Palmer, M. L., Kennedy, P. J. and Noller, H. F. (1978) Complete nucleotide sequence of a 16S ribosomal RNA gene from Escherichia coli. Proc. Natl. Acad. Sci. 75, 480-4805.
Byrant, M. P. (1972) Commentary on the Hungate technique for cultivation of anaerobic bacteria. Am. J. Clin. Nutr. 25, 1324-1328.
Chan, L., Slater, J., Hasbargen, J., Herndon, D. N., Veech, R. L. and Wolf. S. (1994). Neurocardiac toxicity of racemic D, L-lactate fluids. Integr. Physiol. Behav. Sci., 29, 383-394.
Collins, M. D. and Gibson, G. R. (1999). Probiotics, prebiotics, and synbiotics: approaches for modulating the microbial ecology of the gut. Am. J. Clin. Nutr., 69 (suppl), 1052S-1057S.
Counotte, G. H. M., Prins, R. A., Janssen, R. H. A. M., DeBie, M. J. A. (1981). Role of Megasphaera elsdenii in the fermentation of DL-[2-¹³C] lactate in the rumen of dairy cattle. Appl. Environ. Microbiol. 42: 649-655.
Csordas, A. (1996). Butyrate, asprin, and colorectal cancer. Europ. J. Cancer Prevent., 5, 221-231. Day, A. S. and Abbott, G. D. (1999). D-lactic acidosis in short bowel syndrome. New Zealand Med. J. 112: 277-278.
Duncan, S. H., Hold, G. L., Barcenilla, A., Stewart, C. S. and Flint, H. J. (2002). Roseburia intestinalis sp. nov., a novel saccharolytic, butyrate producing bacterium from human faeces. Int. J. System. Evol. Microbiol., 52, 1-6.
Gilmour, M., Flint, H. J. and Mitchell, W. J. (1994). Multiple lactate-dehydrogenase activities of the rumen bacterium Selenomonas ruminantium. Microbiol., 1440, 2077-2084.
Hold, G. L., Pryde, S. E., Russell, V. J., Furrie, E. and Flint, H. J. (2002). The assessment of microbial diversity in human colonic samples by 16S rDNA sequence analysis. FEMS Microbiol. Ecol., 39, 33-39.
Hove, H., Nordgraard-Andersen, I. and Mortensen, B. (1994). Faecal DL-lactate concentration in 100 gastrointestinal patients. Scand. J. Gastroenterol., 29, 255-259.
Hove, H., Holtug, K., Jeppesen, P. B. and Mortensen, P. B. (1995). Butyrate absorption and lactate secretion in ulcerative colitis. Dis. Colon Rect., 38, 519-525.
Inan, M. S., Rasoulpour, R. J., Yin, L., Hubbard, A. K., Rosenberg, D. W. and Giardina, C. (2000). The luminal short-chain fatty acid butyrate modulates NF kappa B activity in a human colonic epithelial cell line. Gastroenterol. 118: 724-734.
Kanauchi, O., Fujiyama, Y., Mitsuyama, K., Araki, Y., Ishii, T., Nakamura, T., Hitomi, Y., Agata, K., Saiki, T., Andoh, A., Toyonaga, A., and Bamba, T. (1999). Increased growth of Bifidobacterium and Eubacterium by germinated barley foodstuff, accompanied by enhanced butyrate production in healthy volunteers. Int. J. Mol. Med., 3, 175-179.
Kung, L. M. and Hession, A. O. (1995). Preventing in vitro lactate accumulation in ruminal fermentations by inoculation Megasphaera elsdenii. J. Anim. Sci., 73, 250-256.
MacFarlane, G. T. and Englyst, H. N. (1986) Starch utilisation by the human large intestinal microflora. J. Appl. Bacteriol. 60, 195-201.
Nocek, J. E. (1997). Bovine acidosis: implications on laminitis. J. Dairy Sci., 80, 1005-1028.
Ouwerkerk, D., Klieve, A. V. and Forster, R. J. (2002). Enumeration of Megasphaera elsdenii in rumen contents by real-time Taq nuclease assay. J. Appl. Microbiol., 92, 753-758.
Pryde, S. E., Duncan, S. H., Hold, G. L., Stewart, C. S. and Flint, H. J. (2002). The microbiology of butyrate formation in the human colon. FEMS Microbiol. Letts., 217, 133-139.
Russell, J. B. and Rychlik, J. L. (2001). Factors that alter rumen microbial ecology. Science, 292, 1119-1122.
Salyers, A. A., West, S. E. H., Vercelloti, J. R. and Wilkins, T. D. (1977) Fermentation of mucus and plant poilysaccharides by anaerobic bacteria from the human colon. Appl. Environ. Microbiol. 34, 529-533.
Schwiertz, A., Hold, G. L., Duncan, S. H., Gruhl, B., Collin, M. D., Lawson, P. A., Flint, H. J. and Blaut, M. (2002). Anaerostipes caccae gen. nov., sp. nov., a new saccharolytic, acetate-utilising, butyrate-producing bacterium from human faeces. Syst. Appl. Microbiol., 25, 46-51.
Suau, A., Bonnet, R., Sutren, M., Godon, J. J., Gibson, G. R., Collins, M. D. and Dore, J. (1999). Direct analysis of genes encoding 16S rRNA from complex communities reveals many novel molecular species within the human gut. Appl. Environ. Microbiol., 65, 4799-4807.
Topping, D. L. and Clifton P. M. (2001) Short chain fatty acids and human colonic function : roles of resistant starch and nonstarch polysaccharides. Physiol. Revs. 81, 1031-1064.
Trinder, P. (1969) Determination of glucose in blood using glucose oxidase with an alternative oxygen acceptor. Ann. Clin. Biochem. 6, 24/27.
Ushida, K., Hashizume, K., Tsukahara, T., Yamada, K. and Koyamai K. (2002). Megasphaera elsdenii JCM ₁₇₇₂^{T} regulates hyper lactate production in the rat large intestine. Reprod. Nutr. Develop., 42 (Suppl. 1) S56-S57.
Weisburg, M. J., Miller, T. L., Yerry, S., Zhang, Y. C. Bank, S. and Weaver, G. A. (1991) Changes of fermentation pathways of faecal microbial communities associated with a drug treatment that increases dietary starch in the human colon. Appl. Environ. Microbiol. 65, 2807-2812.
Wiryawan, K. G. and Brooker, J. D. (1995). Probiotic control of lactate accumulation in acutely grain-fed sheep. Austral. J. Agric. Res., 46, 1555-1568.

1. A method of selecting a strain of lactic acid-utilising bacteria, which method comprises the steps of:
a) providing a bacterial culture from a human faecal sample;
b) selecting a single colony of bacteria;
c) growing said colony in a suitable medium containing lactic acid; and
d) selecting a strain of bacteria consuming relatively large amounts of lactic acid, all of the above steps being conducted under anaerobic conditions.

2. The method as claimed in claim 1 wherein at least 10 mM of lactic acid is consumed during growth into the stationary phase per 24 hours at 37° C. in YCFALG or YCFAL medium.

3. The method as claimed in claim 1 wherein said method comprises the additional step of:
e) selecting a strain of bacteria producing relatively large quantities of butyric acid.

4. The method as claimed in claim 3 wherein at least 10 mM butyric acid is produced during bacterial growth into the stationary phase per 24 hours at 37° C. in YCFALG or YCFAL medium.

5. The method as claimed in claim 1 wherein said lactic acid is a mixture of D and L isomers of lactic acid.

6. Anaerostipes caccae strain L1-92 deposited at NCIMB under No. 13801^{T}.

7. Clostridium indolis bacterial strain Ss2/1 deposited at NCIMB under No. 41156.

8. Eubacterium hallii strain SM 6/1 deposited at NCIMB under No. 41155.

9. A lactic acid utilising bacterium having a 16S rRNA gene sequence with at least 95% homology to one of the sequences shown in FIG. 1.

10. (canceled)

11. (canceled)

12. A method to promote butyric acid formation in the intestine of a mammal, said method comprising the administration of a therapeutically effective dose of at least one strain selected from the group consisting of Anaerostipes caccae strain L1-92 deposited at NCIMB under No. 13801^{T}, Clostridium indolis bacterial strain Ss2/1 deposited at NCIMB under No. 41156 and Eubacterium hallii strain SM 6/1 deposited at NCIMB under No. 41155.

13. The method of claim 12 wherein said bacteria is administered as a foodstuff or as a suppository.

14. A method for treating a disease associated with a high dosage of lactic acid, which method comprises the administration of a therapeutically effective dose of at least one strain of live lactic acid utilising bacteria selected from the group consisting of Anaerostipes caccae strain L1-92 deposited at NCIMB under No. 13801^{T}, Clostridium indolis bacterial strain Ss2/1 deposited at NCIMB under No. 41156 and Eubacterium hallii strain SM 6/1 deposited at NCIMB under No. 41155.

15. The method of claim 14 wherein said disease is lactic-acidosis, short bowel syndrome or inflammatory bowel disease.

16. The method of claim 14 wherein said bacteria is Anaerostipes caccae strain L1-92 deposited at NCIMB under No. 13801^{T}.

17. A prophylactic method to reduce the incidence or severity of colorectal cancer or colitis in mammals caused in part by high lactic acid and low butyric acid concentrations, which method comprises the administration of a therapeutically effective dose of at least one strain of live lactic acid utilising bacteria selected from the group consisting of Anaerostipes caccae strain L1-92 deposited at NCIMB under No. 13801^{T}, Clostridium indolis bacterial strain Ss2/1 deposited at NCIMB under No. 41156 and Eubacterium hallii strain SM 6/1 deposited at NCIMB under No. 41155.

18. The method of claim 17 wherein said bacteria is Anaerostipes caccae strain L1-92 deposited at NCIMB under No. 13801^{T}.

19. A probiotic composition comprising a live bacterial strain selected from the group consisting of Anaerostipes caccae strain L1-92 deposited at NCIMB under No. 13801^{T}, Clostridium indolis bacterial strain Ss2/1 deposited at NCIMB under No. 41156 and Eubacterium hallii strain SM 6/1 deposited at NCIMB under No. 41155, in combination with live lactic acid producing bacteria.

20. The composition as claimed in claim 19 wherein said lactic acid producing bacteria is Lactobacillus spp, Bifidobacterium spp or a mixture thereof.

21. The composition of claim 19 wherein said bacteria is Anaerostipes caccae strain L1-92 deposited at NCIMB under No. 13801^{T}.

22. The composition as claimed in claim 19 further containing other additives or growth enhancing supplements.

23. The method of claim 15 wherein said bacteria is Anaerostipes caccae strain L1-92 deposited at NCIMB under No. 13801^{T}.

24. The method of claim 20 wherein said bacteria is Anaerostipes caccae strain L1-92 deposited at NCIMB under No. 13801^{T}.

25. A method to promote butyric acid formation in the intestine of a mammal, said method comprising the administration of a therapeutically effective dose of at least one lactic acid utilising bacteria according to claim 9.

26. The method of claim 25 wherein said bacteria is administered as a foodstuff or as a suppository.

27. A method for treating a disease associated with a high dosage of lactic acid, which method comprises the administration of a therapeutically effective dose of at least one lactic acid utilising bacteria according to claim 9.

28. The method of claim 27 wherein said disease is lactic-acidosis, short bowel syndrome or inflammatory bowel disease.

29. The method of claim 27 wherein said bacteria is Anaerostipes caccae.

30. The method of claim 28 wherein said bacteria is Anaerostipes caccae.

31. A prophylactic method to reduce the incidence or severity of colorectal cancer or colitis in mammals caused in part by high lactic acid and low butyric acid concentrations, which method comprises the administration of a therapeutically effective dose of at least one strain of live lactic acid utilising bacteria according to claim 9.

32. The method of claim 31 wherein said bacteria is Anaerostipes caccae.

33. A probiotic composition comprising a live bacterial strain as claimed in claim 9, in combination with live lactic acid producing bacteria.

34. The composition as claimed in claim 33 wherein said lactic acid producing bacteria is Lactobacillus spp, Bifidobacterium spp or a mixture thereof.

35. The composition of claim 33 wherein said bacteria is Anaerostipes caccae.

36. The composition of claim 34 wherein said bacteria is Anaerostipes caccae.

37. The composition as claimed in claim 33 further containing other additives or growth enhancing supplements.


SEQUENCE LISTING (10 sequences)

SEQ ID NO: 1 - DNA, 20 bp, Artificial Sequence
  Primer
agagtttgatcmtggctcag

SEQ ID NO: 2 - DNA, 21 bp, Artificial Sequence
  Primer
acggctaccttgttacgactt

SEQ ID NO: 3 - DNA, 18 bp, Artificial Sequence
  Primer
cagcmgccgcggtaatwc

SEQ ID NO: 4 - DNA, 18 bp, Artificial Sequence
  Primer
gwattaccgcggckgctg

SEQ ID NO: 5 - DNA, 20 bp, Artificial Sequence
  Primer
aaactcaaakgaattgacgg

SEQ ID NO: 6 - DNA, 20 bp, Artificial Sequence
  Primer
ccgtcaattcmtttragttt

SEQ ID NO: 7 - DNA, 1450 bp, Eubacterium hallii (Strain D6 1L/1)
gatgaacgctggcggcgtgcctaacactgcaagtcgaacgaagcaccttacctgattcttcggatgaaggtctggtgactgagtggcggacgggtgagtaacgcgtgggtaacctgccctgtacagggggataacagttggaaacggctgctaataccgcataagcgcacgagaggacatcctcttgtgtgaaaaactccggtggtacaggatgggcccgcgtctgattagctggttggcagggtaacggcctaccaaggcgacgatcagtagccggtctgagaggatgaacggccacattggaactgagacacggtccaactcatacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgcatagctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatccttctgaccactccgtaatgggagtcttccttcgggacagaagagacaggtggtgcatggttgtcgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacaaagtgaggcgaacctgcgagggggagcaaaccacaaaaaggccgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagccagtgacccaaccatatggagggagctgtcgaaggtggagccggtaactggggtg

SEQ ID NO: 8 - DNA, 1458 bp, Eubacterium hallii (Strain SM 6/1)
  misc_feature (1263)...(1263): n = a, t, c or g
  misc_feature (1427)...(1427): n = a, t, c or g
  misc_feature (1455)...(1456): n = a, t, c or g
gatgaacgctggcggcgtgcctaacacatgcaagtcgaacgaagcaccttacgagattcttcggatgatcgtttggtgactgagtggcggacgggtgagtaacgcgtgggtaacctgccctgtacagggggataacagctggaaacggctgctaataccgcataagcgcacgaggagacatctcctagtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatagatatggagcaagcgttatccggatttactgggtgtaaagggtgcgtaggtggcagtgcaagtcagatgtgaaaggccggggctcaaccccggagctgcatttgaaactgcwyrgctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggctccggtgccgccgctaacgcagtaagtattccacctggggagtacgttcgcaagaatgaaactcaaaggaattgacggggacccgcacaagcggtggagcatgtggtttaattcgaagcaacgcgaagaaccttaccaggtcttgacatccttctgaccgcaccttaatcggtgctttccttcgggacagaagagacaggtggtgcatggttgtcgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacagagtgaggcgaacccgcgagggggagcaaaccacaaaaaggccgtcccagtncggactgtagtctgcaacccgactacacagaagctggaatcgctagtaatcgcgaatcagaatgtcgcggtgaatacgttcccgggtcttgtacacaccgcccgtcacaccatgggagtcggaaatgcccgaagccagtgacccaacctttatgaaggaagccngtccaaggttgaacccgttaactggggnntt

SEQ ID NO: 9 - DNA, 1477 bp, Ruminococcus gnavus
gagtttgatcctggctcaggatgaacgctggcggcgtgcctaacacatgcaagtcgaacgaggtatattgaattgaagttttcggatggatttcaatgataccgagtggcggacgggtgagtaacgcgtgggtaacctgcctcatacagggggataacggttagaaatgactgctaataccgcataagcgcacagtaccgcatggtacggtgtgaaaaactccggtggtatgagatggacccgcgtctgattagctagttggtggggtaacggcccaccaaggcgacgatcagtagccgacctgagagggtgaccggccacattgggactgagacacggcccagactcctacgggaggcagcagtgggggatattgcacaatggaggaaactctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctgaagtgaaatacccgggctcaacctgggaactgctttggaaactgtgttgctagagtgctggagaggtaagcggaattcctagtgtagcggtgaaatgcgtagatattaggaagaacaccagtggcgaaggcggcttactggacagtaactgacgttgaggctcgaaagcgtggggagcaaacaggattagataccctggtagtccacgccgtaaacgatgaatactaggtgttggtgagcaaagctcatcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaaaaryccyttaatcggrttcctccttcgggacagaggtgacaggtggtgcatggttgtcgtcagctcgtgtcgtgagatgttgggttaagtcccgcaacgagcgcaacccctattgtcagtagccagcaggtgaagctgggcactctgatgagactgccagggataacctggaggaaggtggggatgacgtcaaatcatcatgccccttatgatttgggctacacacgtgctacaatggcgtaaacaaagagaagcgagcctgcgagggggagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcggaaatgcccgaagccagtgaacccaatgcgaaagcagggagctgtcgaaggcaggtctgataactggggtg

SEQ ID NO: 10 - DNA, 1493 bp, Clostridium indolis
agagtttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaactcaaaggaattgacggggacccgcacaagcggtggagcatgtggtttaattcgaagcaacgcgaagaaccttacctggtcttgacatccttctgaccggtccttaaccggacctttccttcgggacaggagtgacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggaggagctgccgaagcgggaccgataactggggtgaagtcgtaaccaggtagccgt